(19) United States
(12) Patent Application Publication
(10) Pub. No.: US 2019/0150521 A1
(43) Pub. Date: May 23, 2019

(54) ELECTRICALLY HEATED UNDERPANTS

(71) Applicant: HSIU-CHU PAI, NEW TAIPEI CITY (TW)

(72) Inventor: HSIU-CHU PAI, NEW TAIPEI CITY (TW)

(21) Appl. No.: 16/185,238

(22) Filed: Nov. 9, 2018

Related U.S. Application Data
(60) Provisional application No. 62/590,279, filed on Nov. 23, 2017.

(51) Int. Cl. A41B 9/04; H05B 1/02; H05B 3/34; A41B 9/00; A41D 13/005; A61F 7/00

(52) U.S. Cl. CPC A41B 9/04; H05B 1/0272; H05B 3/347; A41B 9/001; A41D 13/0051; A61F 7/007; A61F 2007/0071; A41B 2400/32; A41D 2400/10; A41D 2400/32; A61F 2007/0077; A61F 2007/0086; H05B 2203/036

(57) ABSTRACT

Electrically heated underpants are revealed. The electrically heated underpants include an underwear main body, at least one pocket arranged at an outer surface of a front side or a rear side of the underwear main body, and at least one piece of electrically heated textile mounted into the pocket. The pocket is corresponding to abdomen or a lower back of users. The electrically heated textile generates heat and maintains a constant temperature after a current passed therethrough. Thereby the electrically heated underpants can keep users' abdomen and waist warm and further provide health benefits such as keeping the body warm, relieving dull pain in abdomen during the menstrual period, reducing waist and abdominal pain, etc.

### REFERENCE TO RELATED APPLICATION

This Application is based on Provisional Application Ser. No. 62/590,279, filed 23 Nov. 2017, currently pending.

### BACKGROUND OF THE INVENTION

### Field of the Invention

The present invention relates to electrically heated underpants, especially to electrically heated underpants that include at least one piece of electrically heated textile mounted therein to maintain a constant temperature by heating and provide health benefits such as keeping warmth, relief of dull pain in the abdomen during the period, alleviation of waist and abdominal pain.

### Description of Related Art

The effect of air temperature on human health is significant. People should pay attention to changes in temperature and take corresponding measures for their health. For example, the weather is extremely cold when a cold wave comes through in winder and people wear heavy tops, pants and coats to stay warm when they go out. Yet the heavy clothes have a limited effect and not only mobility is severely restricted, getting dressed/undressed is also difficult.

However, thermal clothing made from thin and light cloth also traps body heat to insulate against cold air effectively due to recent advances in textile technology. A lot of other goods are also used to keep warm such as lighter fuel hand-warmers or air-activated disposable hand warmers. Lighter fuel hand-warmers use lighter fluid and require a means of ignition. These are inconvenient to use and the users are in great danger. The temperature of the lighter fuel hand-warmer is unable to be adjusted. Although the hand warmer requires no fuel, its temperature is also unable to be adjusted. Improper use of the hand warmer is very likely to result in skin burns.

The most effective way to stay warm is to raise the body temperature. Besides eating hot food that is high in calories in cold weather, users can also wear undergarments (such as thermal clothing) to prevent heat escaping from inside to outside and provide additional warmth. Underpants are personal items and their cleanness is closely related to user's health, especially for women. Menstruation is a physiological phenomenon in most of women's lifetime. A lot of women suffer from menstrual pain (also called period pain). During menstruation, frequent and sustained contraction of uterine muscles leads to constricted blood supply of the tissue. Thus ischemic pain is induced. In practice, the menstrual cramps can be alleviated by applying heat to the lower abdomen and the temperature of the hot compress is ranging from 39° C. to 40° C. The pain relief effect of the hot compress is as good as the analgesic drugs. Moreover, the physical therapy achieves the pain reduction, without side effects from the drugs.

As to a heat pack or a hot water bottle used for applying heat, it has certain problems such as large volume, heavy weight, etc. more or less. While in use, they are too heavy to use conveniently. There are various portable warmers available on the market such as air-activated disposable hand warmers, lighter fuel hand-warmers or electric heaters. Among them, the air-activated disposable hand warmers are the most commonly used. As to the lighter fuel hand-warmers or the electric heaters, they are too heavy and too large for users to carry with them. Moreover, the lighter fuel hand-warmer has safety problems due to the nature of the fuel. Although the air-activated disposable hand warmers don't have the fuel problem, they still have shortcomings of non-adjustable temperature and skin burns.

Thus there is room for improvement and there is need to provide a novel design that overcomes the above shortcomings.

### SUMMARY OF THE INVENTION

Therefore it is a primary object of the present invention to provide electrically heated underpants that maintain a constant temperature by heat generated therefrom for providing health benefits such as keeping warm, reducing dull pain in abdomen during the menstrual period or alleviating waist and abdominal pain.

In order to achieve the above object, electrically heated underpants according to the present invention mainly include an underwear main body, at least one pocket, and at least one piece of electrically heated textile. The pocket is arranged at an outer surface of a front side or a rear side of the underwear main body and is corresponding to abdomen or a lower back of the user. The electrically heated textile is mounted into the pocket through an opening end of the pocket. Then the electrically heated textile generates heat and maintains a desired temperature after a current passed therethrough. Thereby the electrically heated underpants can be not only used to warm abdomen and waist, buy also help in keeping the body warm, relieving dull pain in abdomen during the menstrual period or reducing waist and abdominal pain.

Preferably, the electrically heated underpants further include a power-supply control unit that not only provides power the electrically heated textile requires but also controls temperature of the electrically heated textile through electronic circuit therein. The temperature can be adjusted within the range of about 20° C. to 45° C.

Preferably, the pocket consists of a first space used for mounting the electrically heated textile and a second space used for mounting the power-supply control unit. The power-supply control unit is electrically connected to the electrically heated textile for providing power to the electrically heated textile.

Preferably, a locking member is disposed on the opening end of the first space and the opening end of the second space respectively. The locking member is selected from the group consisting of a zipper set, a hook-and-loop fastener, or a hanging piece connected to the underwear main body and covering the opening end.

Preferably, the hanging piece and the pocket are arranged with hook-and-loop fasteners or buttons for positioning the hanging piece.

Preferably, the electrically heated textile is formed by interlaced warp wires and weft wires and is composed of a non-conductive area located on a middle part thereof and two conductive areas located on two sides of the non-conductive area. A plurality of insulated wires is set on the non-conductive area while the conductive area is arranged with a plurality of metal wires. A plurality of conductive wires is disposed on a crosswise area while the conductive wires are woven and crossed with the insulated wires and the metal wires.

Preferably, the conductive wire includes a yarn and a metal thread wound around the yarn.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is an explosive view of an embodiment according to the present invention;

FIG. 2 is a schematic drawing showing an embodiment according to the present invention;

FIG. 3 is a schematic drawing showing another embodiment according to the present invention;

FIG. 4 is a schematic drawing showing a further embodiment according to the present invention;

FIG. 5 is a schematic drawing showing a piece of electrically heated textile of an embodiment according to the present invention.

### DETAILED DESCRIPTION OF THE PREFERRED EMBODIMENT

In order to learn technique features and functions of the present invention clearly and completely, please refer to the following detailed description, related figures and reference numbers in the figures.

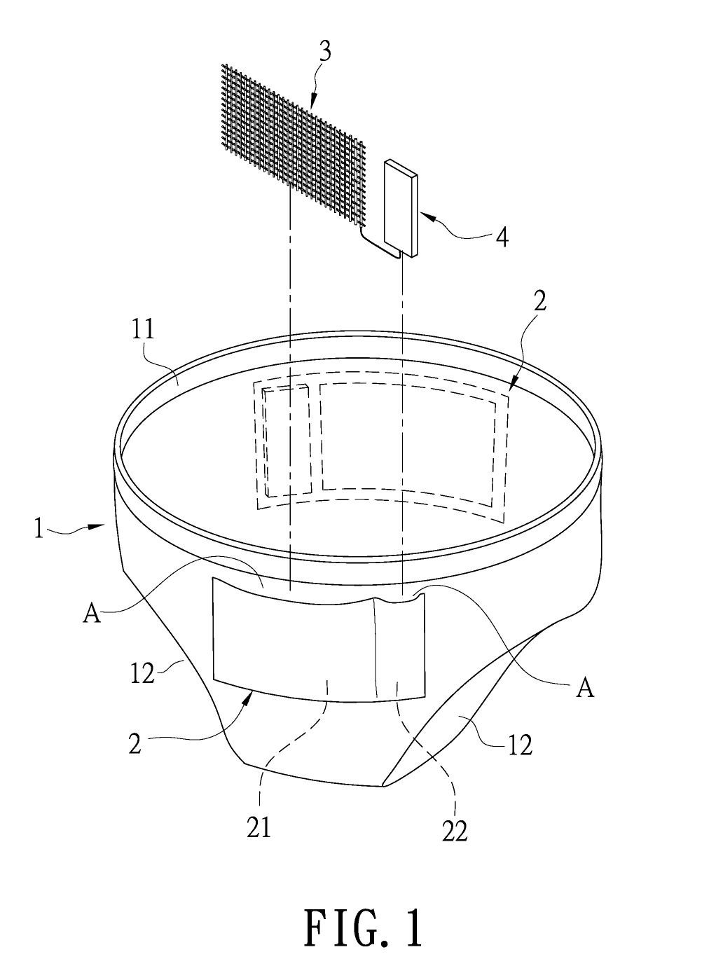

As shown in FIG. 1, electrically heated underpants according to the present invention mainly includes an underwear main body 1, at least one pocket 2 arranged at an outer surface of one side of the underwear main body 1, at least one piece of electrically heated textile 3, and at least one power-supply control unit 4.

The underwear main body 1 consists of a belt portion 11 and two leg openings 12 allowing two legs to pass therethrough respectively. The pocket 2 is disposed on an outer surface of a front side of the underwear main body 1 and corresponding to abdomen, or arranged at an outer surface of a rear side of the underwear main body 1 and corresponding to lower back of user's body. The pocket 2 is composed of a first space 21 and a second space 22 separated from each other. The first space 21 as well as the second space 22 has an opening end (A).

The electrically heated textile 3 is mounted in the first space 21 of the pocket 2.

The power-supply control unit 4 is set in the second space 22 of the pocket 2 and is electrically connected to the electrically heated textile 3 for providing power to the electrically heated textile 3.

The materials, the shape and the style of the underwear main body 1 are not limited. The underwear main body 1 can be made from various kinds of fibers. The underwear main body 1 can be designed into men's or women's underpants. The underwear main body 1 includes the belt portion 11 worn around the user's waist and two leg openings 12 allowing the user's legs to pass therethrough respectively. The underwear main body 1 can be integrally produced or formed by a plurality piece of fiber cloth sewn together.

During manufacturing, refer from FIG. 1 to FIG. 4, the pocket 2 is arranged at the outer surface of the front side, the rear side or both the front and the rear sides of the underwear main body 1. The pocket 2 can be connected to the underwear main body 1 by sewing. The space inside the pocket 2 is divided into two spaces including the first space 21 and the second space 22. The size of the first space 21 and the second space 22 can be modified according to user's needs. Generally, the first space 21 is larger than the second space 22. This is owing to that the power-supply control unit 4 is usually compact for user's comfort. Thus the second space 22 required for mounting the power-supply control unit 4 is not large.

The users can select the way they want to use according to their health conditions or the weather condition while using the electrically heated underpants. For example when a cold current comes, the user can use the electrically heated underpants for warming both abdomen and lower back. When a female is in her period, she can use the electrically heated underpants for warming only the abdomen. The user can also use the electrically heated underpants for warming the lower back when his/her waist and back feel achy. After wearing the underwear main body 1, the electrically heated textile 3 is mounted in the first space 21 of the pocket 2 through the opening end (A). Then the electrically heated textile 3 is electrically connected to the power-supply control unit 4 to get power required. The power-supply control unit 4 is mounted into the second space 22 of the pocket 2 through the opening end (A), not only used to provide power to the electrically heated textile 3, but also to control temperature of the electrically heated textile 3 by electronic circuit therein. The temperature can be adjusted within the range of around 20 degrees Celsius (° C.) to 45° C. This is the optimal temperature range for warming the abdomen and the lower back of human body, or pain reduction such as relieving dull pains in abdomen during menstruation, alleviating pain caused by uterine contraction and accelerating blood circulation, reducing cramps resulted from contraction of blood vessels, activating the blood circulation for reducing lower back pain, etc.

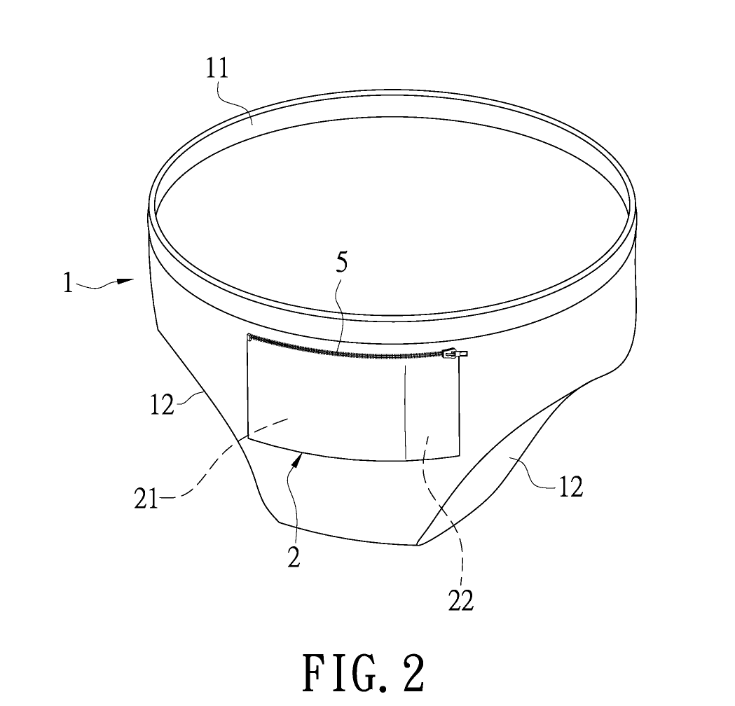

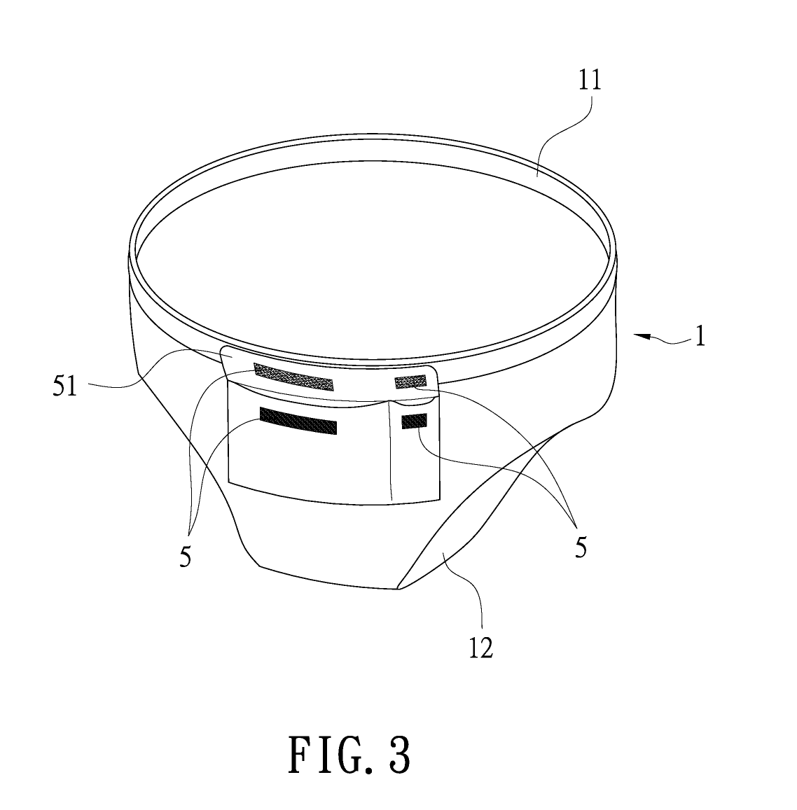

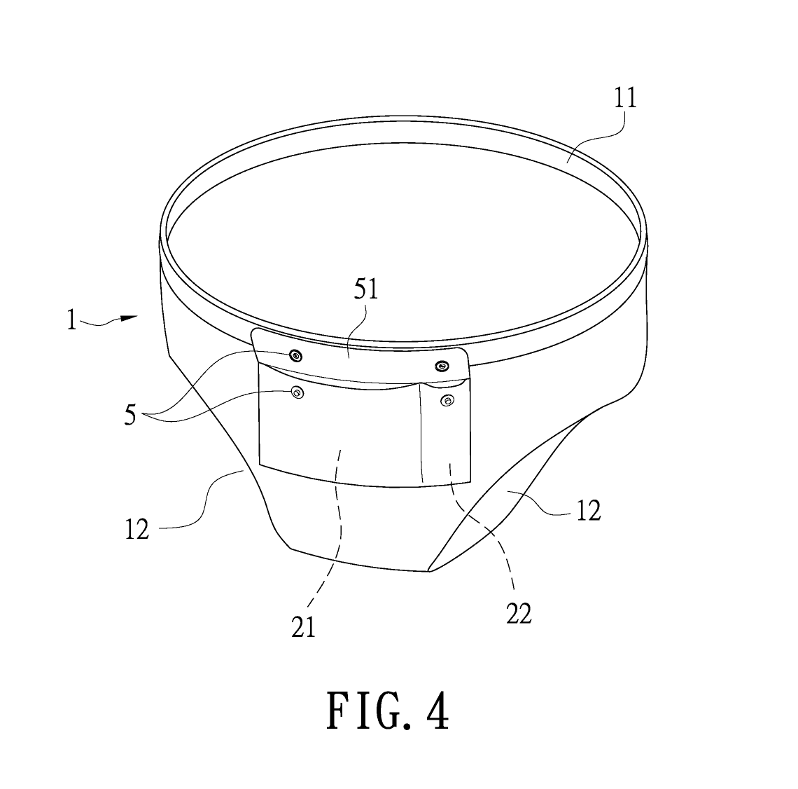

Refer to FIG. 2, FIG. 3 and FIG. 4, in order to allow the electrically heated textile 3, and the at least one power-supply control unit 4 to be firmly positioned in the first space 21 and the second space 22 respectively, a locking member 5 is disposed on the opening end (A) of the first space 21 and the opening end (A) of the second space 22 respectively. The locking member 5 can be a zipper set or a hook-and-loop fastener used for enclosing and limiting the electrically heated textile 3, and the power-supply control unit 4 in the first space 21 and the second space 22 respectively. The hook-and-loop fastener includes two lineal fabric strips that are provided with tiny hooks and small loops respectively and attached to the opposing surfaces to be fastened. The locking member 5 can also be a hanging piece 51 hanging from the top of the pocket 2 and connected to the underwear main body 1 for covering the opening end (A). The hanging piece 51 and the pocket 2 are provided with hook-and-loop fasteners or buttons for positioning the hanging piece 51 so that the electrically heated textile 3 and the power-supply control unit 4 can be enclosed in the first space 21 and the second space 22 by the hanging piece 51 being positioned.

The electrically heated textile 3 generates heat when an electrical current flows therethrough and has a plurality of modes. The following is a feasible mode.

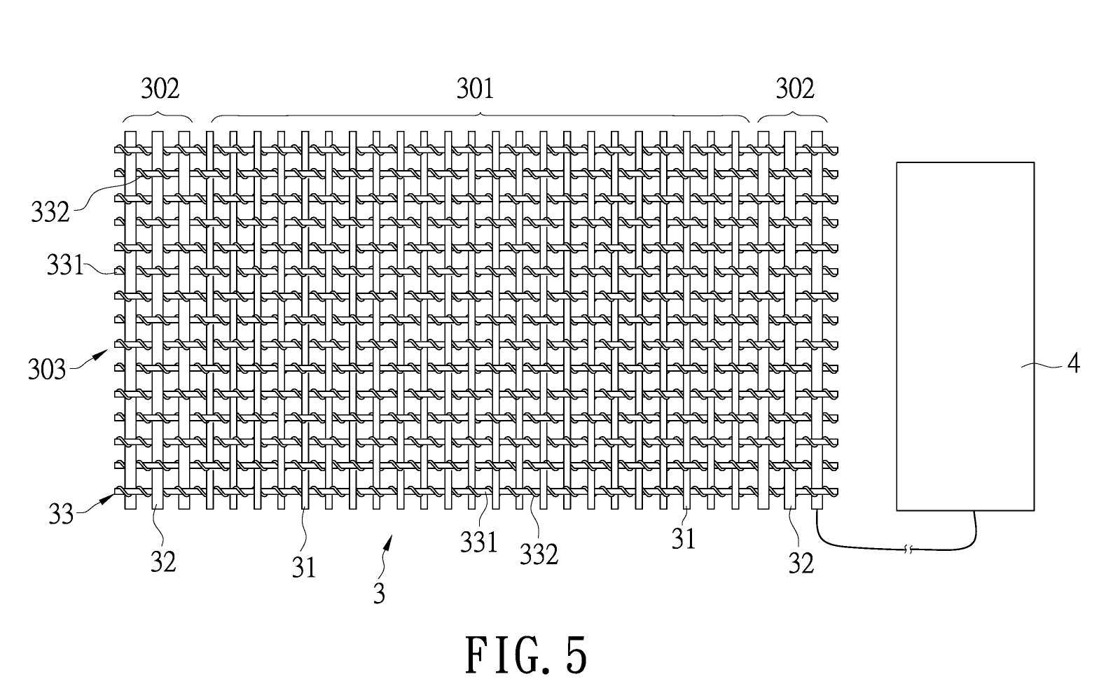

Refer to FIG. 5, the electrically heated textile 3 is formed by warp wires and weft wires crossed each other and is composed of a non-conductive area 301 located on a middle part thereof and two conductive areas 302 located on two sides of the non-conductive area 301. A plurality of insulated wires 31 is arranged at the non-conductive area 301 while the conductive area 302 is provided with a plurality of metal wires 32. A plurality of conductive wires 33 is all disposed on a crosswise area 303 over the length of the electrically heated textile 3. The length is defined as the distance between two sides of the electrically heated textile 3 along the horizontal direction. The conductive wire 33 is formed by a yarn 331 and a metal thread 332 wound around the yarn 331. The metal thread 332 is thread made from metal and having a diameter of 0.01-0.025 mm. The electrically heated textile 3 is formed by the conductive wires 33 woven and crossed with the insulated wires 31 and the metal wires 32.

The size of the electrically heated textile 3 depends on the first space 21. The width of the conductive area 302 on each of two sides of the electrically heated textile 3 is about 1/10 of the total width of the electrically heated textile 3. For example, when the total width of the electrically heated textile 3 is 10 cm, the width of the respective conductive areas 302 is about 1 cm. The width of the non-conductive area 301 is 8 cm. The metal wires 32 of the conductive area 302 are connected to the power-supply control unit 4 for generating heat when a current is flowing through the metal wire 32 with high electrical resistance. Moreover, the electrically heated textile 3 is formed by woven wires so that it is soft, washable, rollable, foldable, convenient to use and easy to store. While being mounted into the first space 21 of the pocket 2 of the underwear main body 1, the electrically heated textile 3 is attached to and moved along with the user so that the user will not feel weird and discomfort.

In summary, the electrically heated underpants according to the present invention have the following advantages compared with the structure available now.
1. The electrically heated textile mounted in the pocket of the electrically heated underpants provides heat to the user's abdomen or waist properly to keep these areas warm, relieving dull pains in abdomen during menstruation, alleviating pain caused by uterine contraction and accelerating blood circulation, reducing cramps resulted from contraction of blood vessels, or activating the blood circulation for reducing lower back pain, etc.
2. The electrically heated textile is cloth formed by woven wires so that it is soft and flexible, washable, rollable, foldable, convenient to use and easy to store. The electrically heated textile is attached to and moved along with the user so that the user feels comfortable while wearing the electrically heated underpants, without feeling weird.
3. Besides providing the power required by the electrically heated textile, the power-supply control unit also controls the temperature of the electrically heated textile to maintain a constant temperature within a temperature range of 20° C.-45° C. for protecting users from being burned.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

What is claimed is:

1. Electrically heated underpants comprising:
an underwear main body that includes a belt portion and two leg openings allowing two legs to pass therethrough respectively;
at least one pocket that is disposed on an outer surface of at least one side of the underwear main body, and is corresponding to abdomen or/and lower back of human body; the pocket including a first space and a second space separated from each other and having an opening end respectively;
at least one piece of electrically heated textile mounted in the first space of the pocket; and
at least one power-supply control unit that is set in the second space of the pocket and is electrically connected to the electrically heated textile for providing power to the electrically heated textile.

2. The device as claimed in claim 1, wherein the power-supply control unit controls temperature of the electrically heated textile through electronic circuit therein and the temperature is able to be controlled and adjusted within a range of 20° C. to 45° C.

3. The device as claimed in claim 1, wherein a locking member is disposed on the opening end of the first space and the opening end of the second space respectively; the locking member is selected from the group consisting of a zipper set and a hook-and-loop fastener.

4. The device as claimed in claim 1, wherein a locking member is disposed on the opening end of the first space and the opening end of the second space respectively; the locking member is a hanging piece that is connected to the underwear main body and covering the opening end.

5. The device as claimed in claim 4, wherein the hanging piece and the pocket are provided with hook-and-loop fasteners or buttons for positioning the hanging piece.

6. The device as claimed in claim 1, wherein the electrically heated textile is formed by interlaced warp wires and weft wires and is composed of a non-conductive area located on a middle part thereof and two conductive areas located on two sides of the non-conductive area; a plurality of insulated wires is disposed on the non-conductive area while the conductive area is provided with a plurality of metal wires; a plurality of conductive wires is disposed on a crosswise area, woven and crossed with the insulated wires and the metal wires; the metal wires of the conductive area on each of two sides of the electrically heated textile are electrically connected to the power-supply control unit.

7. The device as claimed in claim 6, wherein the conductive wire includes a yarn and a metal thread wound around the yarn.

8. The device as claimed in claim 6, wherein the metal thread of the conductive wire is a thread made from metal and having a diameter of 0.01-0.025 mm.

9. The device as claimed in claim 7, wherein the metal thread of the conductive wire is a thread made from metal and having a diameter of 0.01-0.025 mm.
